(19) United States
(12) Patent Application Publication
(10) Pub. No.: US 2006/0134230 A1
(43) Pub. Date: Jun. 22, 2006

(54) Weight loss composition and formulation

(76) Inventor: Sal Abraham, Easton, PA (US)

Correspondence Address: SAL ABRAHAM, 1470 MOHAWK LANE, EASTON, PA, 18040, US

(21) Appl. No.: 10/905,131

(22) Filed: Dec. 17, 2004

(51) Int. Cl. A61K 36/898; A61K 36/82

(52) U.S. Cl. 424/725; 424/729; 424/769

(57) ABSTRACT

This invention discloses a new and unique combination of Dahurian Angelica Root extract, Polygonum multiflorum extract, Theobroma cocoa extract, Yerba mate extract, Oolong tea extract, Kohki Tea extract, Horse Chesnut extract, and Ligustrum Lucidum extract useful as a dietary supplement for increasing weight loss in humans.

### BACKGROUND OF INVENTION

Over recent years obesity has reached epidemic proportions. Obesity contributes to 400,000 deaths each year and according to federal guidelines, half the population is overweight and a third is obese. Obesity is defined as an excess proportion of total body fat correlating with a body weight greater than 20 percent of ideal body weight (IBW). Body Mass Index (BMI) is another method to determine whether or not an individual is obese. BMI is calculated with an equation consisting of weight and height measurements in order to determine total body fat. A BMI between 25-29.9 indicates an individual is overweight. Causes for obesity include genetic, environmental, economic, emotional, and physiological factors. These factors can then lead to the over consumption of total calories. The amount of total calories consumed versus the amount of total calories burned determines the amount of fat stored for energy reserves. Calories or Kcals (kilocalories) are defined as the amount of heat necessary to raise the temperature of 1 gram of water 1 degree Celsius. The amount of total calories burned is defined as the calories utilized by exercise plus basal metabolic rate (BMR) or resting metabolic rate (RMR). BMR represents the amount of calories needed to maintain IBW at rest. Increasing BMR results in fewer calories stored as fat and can promote weight loss if the amount calories burned is greater than the amount of calories ingested. One of the main factors that controls BMR is the percentage of lean body weight.

Standard medical therapy for obesity includes oral prescription medications. Most of these medications are designed to regulate appetite by releasing serotonin or catecholamine. For instance Sanorex, Mazanor, Adipex-P, and Meridia are common appetite suppressant medications. However most of these medications can only be used on a short-term basis and are scheduled as controlled substances due to the fact that they can become addictive. Other side effects include increased heart rate, blood pressure, constipation and insomnia. Merida is the only appetite suppressant that has been approved for long-term use. Another long-term pharmaceutical approach to weight loss is the fat absorption inhibitor Xenical. Xenical works by blocking about 30 percent of dietary fat from being absorbed. Enzymes in the digestive system, called lipases, assist in the digestion of dietary fats. Xenical attaches to the lipases and inhibits the digestion of dietary fat as triglycerides into absorbable free fatty acids and monoglycerides, which are then excreted in the bowel. Xenical literature recommends not ingesting more than 30 percent of total calories from dietary fat per day due to concerns regarding loose bowels. It appears that a common and unpleasant side effect of Xenical includes flatulence and loose bowels when consumed with a high fat diet.

U.S. Pat. No. 5,422,352 to Arne Astrup relates a method for a slimming pharmaceutical composition. This invention represents an improvement in standard pharmaceutical weight loss medications due to its non-addictive properties. The use of ephedrine and caffeine has been documented as an effective weight loss preparation. Ephedrine and caffeine produce a thermogenic response and decrease appetite by stimulating the release of catecholamines. Ephedrine is categorized as a sympatomimetic compound, which utilizes the adrenergic alpha and beta-receptors, involved in the release of the catecholamines. Since ephedrine is not a selective beta agonist it stimulates all of the main beta receptors (beta1, beta2, and beta3) resulting in the stimulation of metabolic rate, blood pressure, and heart rate. Although this increase in blood pressure and heart rate is less than ideal in number of individuals.

U.S. Pat. No. 6,316,499 to Dennis Jones relates a method for increasing muscle mass of a human with materials derived from citrus varieties. This invention represents an improvement in standard pharmaceutical and dietary supplement weight loss products due its natural origin and non-addictive properties. Citrus Aurantium contains several alkaloids including hordenine, octopamine, tyramine and N-methyltyraminesynephrine and synephrine as the principal alkaloid. These alkaloids are thought to be beta selective and only stimulate the beta 3 receptor, which is responsible for metabolic rate. This represents an improvement in weight loss therapies. However, depending upon the dose, these alkaloids can stimulate the alpha receptors, which are partly responsible for blood pressure. According to this patent citrus varieties containing these alkaloids can also be used to promote muscle mass in humans when combined with a high protein diet and weight training program. Although while this may be an improvement in standard pharmaceutical weight loss therapy it is still less than desirable due to the possible increase in blood pressure.

U.S. Pat. No. 5,804,596 to Muhammed Majeed et al. relates a method of preparing forskohlin composition from forskohlin extract and use of forskohlin for promoting lean body mass and treating mood disorders. This invention represents a further improvement in standard pharmaceutical and dietary supplement weight loss products due to the fact that it is naturally derived, non-addictive, and a non-stimulant. Forskohlin extract is derived from the Coleus Forskoli plant and a method for this extraction is described. Forskohlin stimulates noradrenaline production, which in turn stimulates the beta adrenergic receptors to produce adenyl cyclase. Adenyl cyclase is responsible for the promotion cAMP (cyclic adenosine monophosphate), which in turn activates protein kinase to phosphorylate HSL (hormone sensitive lipase) for the release of fatty acids from adipose tissue. The increase in cAMP is thought to correspond to enhancing the thermogenic response to food thus improve absorption of nutrients and their incorporation into lean body mass. The increased release of fatty acids and the increase in lean body mass contribute to shifting of the lean body mass/adipose tissue ratio in favor lean body mass for the regulation body weight. Forskohlin also restores the level of monoamines for presynaptic availability, which has known anti-depressant action and which may contribute better eating habits. However, these actions can be easily blunted by an insulin response generated from a meal.

U.S. Pat. No. 6,531,162 to William Llewellyn relates to a non-stimulant composition and formulation for decreasing body weight. The combination of octopamine, yohimbine, bergenin, and decaffeinated green tea extract increases lipolysis, thermogenesis, and weight loss, and is safe and much less apt to cause the side effects normally associated with stimulant-based weight loss products. Octopamine is a naturally occurring catecholamine structurally related to norephinephrine, and has been proven in in-vitro studies to be a potent selective beta-3 agonist. Yohimbine is an extremely potent naturally occurring alpha-2 receptor antagonist. Adrenergic lipolysis in human adipose tissue is regulated in a dual nature by adrenoceptors. Studies with Green Tea extract with standardized amounts of EGCG have suggested it exerts a direct effect on thermogenesis by increasing the respiration rate of brown fat cells, and furthermore that it can strongly enhance the lipolytic action of other chemicals or agents acting on this system. Bergenin has been shown to have an action in the body that augments the lipolytic action of norepinephrine. However, while this may be an improvement in weight loss formulations it is not a truly non-stimulant formula. Octopamine is not a selective beta 3 agonist but actually an alpha-Adrenergic sympathomimetic amine, biosynthesized from tyramine in the central nervous system and platelets and also in invertebrate nervous systems. Yohimbine is also known to increase blood pressure so when combined with octopamine it can result in strong stimulant activity.

### SUMMARY OF INVENTION

This invention discloses a new and unique combination consisting of Dahurian Angelica Root extract, Polygonum multiflorum extract, Theobroma cocoa extract, Yerba mate extract, Oolong tea extract, Kohki Tea extract, Horse Chesnut extract, and Ligustrum Lucidum extract useful as a dietary supplement for increasing weight loss. The problem of the present invention is to provide a composition that decreases body weight with out any of the previously mentioned negative side effects associated with pharmaceutical or nutraceutical compositions. According to the invention these problems are solved by the use of a carefully blended composition containing Dahurian Angelica Root extract, Polygonum multiflorum extract, Theobroma cocoa extract, Yerba mate extract, Oolong tea extract, Kohki Tea extract, Horse Chesnut extract, and Ligustrum Lucidum extract. Prior art also discloses these eight compounds of interest to this inventor, but which heretofore had not been combined to create a new and useful weight loss product.

### DETAILED DESCRIPTION

This invention discloses the formula sets that embody the invention of the supplement composition for increasing weight loss in humans. The combination of Dahurian Angelica Root extract, Polygonum multiflorum extract, Theobroma cocoa extract, Yerba mate extract, Oolong tea extract, Kohki Tea extract, Horse Chesnut extract, and Ligustrum Lucidum extract possess the ability to act as oxidative uncouplers, nitric oxide, fatty acid synthase and anti-lipolytic hormone inhibitors.

The first unique and novel combination is that of Dahurian Angelica Root extract and Polygonum multiflorum extract. Dahurian Angelica Root contains natural coumarins or more specifically furanocoumarins such as imperatorin and psoralen. Furanocoumarins dramatically promote and initiate lipolysis or fat burning. For instance Yoshiyuki et al. Planta Med 1982 July 45:3 183-7 has demonstrated the ability of furanocoumarins to activate the actions of lipolytic hormones and selectively inhibit the effects of anti-lipolytic hormones such as insulin. Furanocoumarins have been shown to activate adrenaline induced lipolysis while at the same time selectively inhibiting the anti-lipolytic hormone insulin.

A vital function of furanocoumarins is there ability to act as oxidative uncouplers. Olorunsogo et al. Chem Biol Interact 1990; 74(3): 263-74 has demonstrated the ability of chalepin, imperatorin and marmesin to act as oxidative uncouplers. Oxidative phosphorylation is the formation of adenosine diphosphate to adenosine triphosphate, which is the primary energy source for many physiological functions. When uncoupling of this reaction occurs, free energy or heat is released resulting in a thermogenic response and caloric expenditure.

Furanocoumarins are inhibitors of the enzyme cAMP phosphodiesterase. This enzyme is responsible for inhibiting lipolysis or fat burning. Sadari et al. Pharmazie 1999 Jul. 54(7):554-6 has demonstrated the ability of furanocoumarins to inhibit cAMP phosphodiesterase and thus enhance lipolysis.

Another interesting function of furanocoumarins is their ability to inhibit nitric oxide production. The inhibition nitric oxide production has been associated with weight loss in animals. Interestingly the nitric oxide biosynthetic pathway is thought to contribute to the regulation of feeding. Inhibition of nitric oxide causes a decrease in appetite and slows gastric emptying. Wang C C et al. Cancer Lett 1999 Oct. 18; 145(1-2):151-7 has demonstrated the ability of furanocoumarins to act as potent nitric oxide inhibitors. Morely, et al. Life Sci 1992; 51(16): 1285-9, Morely et al. Pharmacol Biochem Behav 1995 March; 50(3): 369-73, and Plourde V et al. Eur J Pharmacol 1994 Apr. 21; 256(2): 125-9 clearly demonstrates the role of the nitric oxide inhibition and weight loss. The use of furanocoumarins for weight regulation is somewhat of a new application that has been clearly overlooked for a number of years.

Polygonum multiflorum contains various hydroxy anthraquinones and can be standardized specifically for Emodin (3-methyl-1,6,8-trihydroxy-anthraquinone). Hydroxy anthraquinones are oxidative uncouplers as described by Betina V et al. Chem Biol Interact 1987; 62:1 79-89. Oxidative uncouplers inhibit oxidative phosphorylation or the formation of ATP. This inhibition results heat production and calories burned at rest. Emodin is a hydroxy anthraquinone that acts as an oxidative uncoupler and fatty acid synthase inhibitor. The fatty acid synthase enzyme is involved in the biosynthesis of fat. Emodin has been shown to inhibit or slow down its activity and thus reduce body fat. According to Zhang Chongben et al. Chin Med J 2002; 115(7): 1035-1038 emodin has an inhibition effect on fatty acid synthase, which is dose dependent. The inhibition of differentiation of 3T3-L1 by emodin might be related to the blockage of fatty acid synthase activity, because lipid synthesis is a very important biochemical event that causes the differentiation of preadipocyte into adipocyte. Emodin has an effect on the differentiation and proliferation of preadipocyte, as well as on lipid metabolism. Prior art also discloses these two compounds of interest to this inventor, but which heretofore had not been combined to create a new and useful weight loss combination.

The next combination consists of Theobroma cocoa extract and Yerba mate extract. This combination functions as a lipolysis potentiator and gastric emptying inhibitor. Theobroma coca can be standardized to contain methylxanthines such as theobromine, while Yerba mate can be standardized to contain caffeine and theobromine in addition to glycosides and mate saponins. Methylxanthines promote fat burning via a direct thermogenic response and inhibit the phosphodiesterase enzyme, which mediates the anti-lipolytic action of insulin. The amount of methylxanthine found in this invention is minimal and therefore overcomes the limitations of previous inventions.

Yerba mate formulations have been shown to decrease body weight and gastric emptying resulting in fat loss and appetite suppression. Andersen, T et al. J Hum Nutr Diet. 2001 June; 14(3): 243-50 discloses a herbal preparation containing yerba mate, that significantly delayed gastric emptying, reduced the time to perceived gastric fullness and induced significant weight loss over 45 days in overweight patients treated in a primary health care context.

The next unique and novel combination consists of Oolong tea extract and Kohki tea extract. According to traditional Chinese belief oolong tea is effective in controlling body weight. Rumpler, W. et al. J Nutr 2001 November 131:2848-52 has demonstrated the ability of oolong tea to increase 24 hour energy expenditure in humans. Oolong tea extract can be standardized to contain polyphenols, catechins, and caffeine.

Kohki tea is produced from the leaves of Engelhardtia chrysolepis (Chinese name, huang-qui) and can be standardized to contain the dihydroflavonol taxifolin and its glycoside astilbin. Han L K. et al. J. Nat. Prod. Vol 61 August 1998, P1006-1011, (REF 25) and Motoyashiki, T. et al. Biol Pharm Bull 1998 May; 21(5): 517-9 show that these standardized extracts stimulate adrenocorticotrophic hormone induced lipolysis and inhibit insulin induced lipogenesis from glucose. Prior art also discloses these two compounds of interest to this inventor, but which heretofore had not been combined to create a new and useful weight loss combination.

The final unique and novel combination consists of Horse chestnut extract and Ligustrum licidum extract. This combination delays gastric emptying and glucose absorption and thus results in appetite suppression and less plasma insulin secretion. Horse chestnut extract can be standardized to contain escins and Ligustrum licidum extract can be standardized to contain oleanic acid. Matsuda, H et al. Eur J Pharmacol 1999 Mar. 5; 368(2-3): 237-43 has demonstrated the inhibitory effects of the saponin fraction and its principal constituents, escins Ia, Ib, IIa, and IIb, from horse chestnuts on gastric emptying. Delaying gastric emptying causes a meal to leave the stomach and enter the small intestine over a longer period of time thus increasing satiety. It also slows the digestion of carbohydrate to glucose resulting in less lipogenic insulin release. Matsuda, H et al. Chem Pharm Bull (Tokyo). 1998 September; 46(9): 1399-403 has demonstrated the ability of oleanic acid to suppress the transfer of glucose from the stomach to the small intestine and by inhibiting glucose transport at the brush border of the small intestine. Prior art also discloses these two compounds of interest to this inventor, but which heretofore had not been combined to create a new and useful weight loss combination.

It is now believed that one skilled in the art can, using the following descriptions, can utilize the present invention to its fullest extent. The following examples illustrate a weight loss combination and formulation that is safe and that does not cause any of the previously mentioned negative side effects. The following examples should not be considered as limitations of the present invention.

### EXAMPLE 1

### Weight Loss Combination and Formulation

3 capsules contain:
150 mg Angelica dahurica var. pai-chih extract (Standardized for 5% imperatorin)
150 mg Polygonum multiflorum extract (Standardized for 75% Emodin)
150 mg Theobroma cocoa extract (standardized for 10% theobromine)
150 mg Yerba mate (standardized for 20% caffeine, 7% theobromine, 7% Glycosides, 2% Mate saponin)
150 mg Oolong Tea extract (standardized for 40% polyphenol, 20% catechin, and 9% caffeine)
150 mg Kokhi tea extract (standardized for 10% astilbin and taxifolin)
550 mg Horse Chesnut (standardized for 20% escins)
550 mg Ligustrum lucidum (standardized for 98% Oleanolic Acid)
Excipients include dicalcium phosphate and magnesium stearate for suitable encapsulation

### EXAMPLE 2

### Weight Loss Combination and Formulation

3 capsules contain:
300 mg Angelica dahurica var. pai-chih extract (Standardized for 5% imperatorin)
300 mg Polygonum multiflorum extract (Standardized for 75% Emodin)
150 mg Theobroma cocoa extract (standardized for 10% theobromine)
150 mg Yerba mate (standardized for 20% caffeine, 7% theobromine, 7% Glycosides, 2% Mate saponin)
150 mg Oolong Tea extract (standardized for 40% polyphenol, 20% catechin, and 9% caffeine)
150 mg Kokhi tea extract (standardized for 10% astilbin and taxifolin)
Excipients include dicalcium phosphate and magnesium stearate for suitable encapsulation

### EXAMPLE 3

### Weight Loss Combination and Formulation

2 capsules contain:
300 mg Angelica dahurica var. pai-chih extract (Standardized for 5% imperatorin)
300 mg Polygonum multiflorum extract (Standardized for 75% Emodin)
150 mg Theobroma cocoa extract (standardized for 10% theobromine)
150 mg Yerba mate (standardized for 20% caffeine, 7% theobromine, 7% Glycosides, 2% Mate saponin)
Excipients include dicalcium phosphate and magnesium stearate for suitable encapsulation

### EXAMPLE 4

### Weight Loss Combination and Formulation

2 capsules contain:
500 mg Angelica dahurica var. pai-chih extract (Standardized for 5% imperatorin)
500 mg Polygonum multiflorum extract (Standardized for 75% Emodin)
Excipients include dicalcium phosphate and magnesium stearate for suitable encapsulation

The foregoing descriptions of the invention are for illustration only. Modifications not included in the description, which are obvious to those skilled in the art, are intended to be included in the scope of the following claims.

1-4. (canceled)

5. A weight loss method comprising administering a composition of matter comprising Angelica Dahurica (Fisch ex Hoffm.) Benth et. Hook f. and Polygonum cuspidatum Sieb. et Zucc. in amounts effective to promote weight loss.

6. The composition according to claim 5, which further comprises Theobroma Cacao L., llex Paraguayensis, Camellia sinensis (L.) Kuntze or Engelhardtia chrysolepis.

7. The composition according to claim 5, which further comprises Aesculus Hippocastanum L. or Ligustrum Lucidum.

8. A weight loss method comprising administering a composition of matter comprising imperatorin and emodin in amounts effective to promote weight loss.

9. The composition according to claim 8, which further comprises theobromine, caffeine, mate saponin, caffeic acid, polyphenols, catechins, astibilin or taxifolin.

10. The composition according to claim 8, which further comprises escins or oleanic acid.
